(19) United States
(12) Patent Application Publication
(10) Pub. No.: US 2025/0288242 A1
(43) Pub. Date: Sep. 18, 2025

(54) MINIMAL INVASIVE ELECTROCORTICOGRAPHIC BRAIN-COMPUTER INTERFACE

(71) Applicants: The University of Hong Kong, Hong Kong (CN); Advanced Biomedical Instrumentation Centre Limited, Hong Kong (CN)

(72) Inventors: Kwok Leung CHAN, Hong Kong (CN); Tao ZOU, Hong Kong (CN); Na XIAO, Hong Kong (CN)

(21) Appl. No.: 19/072,514

(22) Filed: Mar. 6, 2025

Related U.S. Application Data
(60) Provisional application No. 63/564,204, filed on Mar. 12, 2024.

(51) Int. Cl. A61B 5/37; A61B 5/268; A61B 5/293; A61B 5/386; A61B 17/00

(52) U.S. Cl. CPC A61B 5/37; A61B 5/268; A61B 5/293; A61B 5/386; A61B 17/00234; A61B 2017/00318

(57) ABSTRACT

A guidewires-driven brain-computer interface system for minimally invasive implantation is provided, including an array of flexible electrodes and a plurality of flexible guidewires attached to the array of flexible electrodes. The array of flexible electrodes includes a plurality of insulation parylene-C layers, metal interconnect lines, and electrodes. The plurality of flexible guidewires are configured to be guided by an external mechanical force such that the array of flexible electrodes traverse craniotomy and are unfurled by tracking the plurality of flexible guidewires. The flexible guidewires each includes a steering tip and a pulling thread.

### CROSS-REFERENCE TO RELATED APPLICATION

The present application claims the benefit of U.S. Provisional Application Ser. No. 63/564,204, filed Mar. 12, 2024, which is hereby incorporated by reference herein in its entirety, including any figures, tables, or drawings.

### BACKGROUND OF THE INVENTION

Bidirectional brain-computer interface (BCI) has significantly contributed to both fundamental neuroscience research and clinical applications, encompassing areas such as visual₁₋₄, sensorimotor^{5,6}, and auditory neuroprostheses^{7,8}, as well as the detection and treatment of epilepsy^{9,10} and Parkinson's disease¹¹. Among these, Electrocorticographic brain-computer interface (ECoG-BCI) employ surface electrode implants to harvest neural signals from the brain's surface^{12,13}. Compared with scalp electroencephalography and intraparenchymal single neuronal recordings, ECoG-BCI provides an optimal compromise between signal acquisition and surgical invasiveness, thus becoming a gold standard for clinical data collection and human subject research in neuroscience¹⁴⁻¹⁶.

Enhancing the recording scope and density of ECOG-BCI is crucial for analyzing the interconnected activities of multiple brain regions¹⁷, improving signal decoding accuracy^{18,19}, and precisely identifying neural disease foci²⁰. Traditionally, the implantation of large-area coverage ECoG-BCI commonly necessitates an equally substantial cranial opening^{21,22}, which would expose significant brain tissue to the external environment. This could cause brain tissue swelling²³, inflammation²⁴, heighten risk of cortical damage²⁵, and infection²⁶, thereby deferring acceptance among potential BCI users and patients^{27,28}.

To mitigate these risks and extend the benefits of ECOG-BCIs to a broader patient and user base, various minimally invasive surgical techniques have been developed. These include pressure-driven actuation²⁹, magnetic force-assisted positioning³⁰, cranial micro-slit insertion³¹, and shape memory alloy actuation³². These reported methods can deploy a larger device than the opened skull holes^{29,30,32} or implant a high density device through a cranial slit³¹. However, a method that achieves both extensive coverage and high density with minimal invasiveness has yet to be reported.

### BRIEF SUMMARY OF THE INVENTION

There continues to be a need in the art for improved designs and techniques for a minimal invasive electrocorticographic brain-computer interface.

According to an embodiment of the subject invention, a guidewires-driven brain-computer interface (GD-BCI) system for minimally invasive implantation is provided. The system comprises an array of flexible electrodes and a plurality of flexible guidewires attached to the array of flexible electrodes. The array of flexible electrodes comprises a plurality of insulation parylene-C layers, metal interconnect lines, and electrodes made of PEDOT:PSS/pHEMA and gold. The adjacent metal interconnect lines are spaced apart by a distance of 10 μm. The metal interconnect lines are formed of gold and fully encapsulated between adjacent insulation parylene-C layers. Moreover, the array of flexible electrodes has a density of 28.4 electrodes per cm². Furthermore, the array of flexible electrodes is formed by biocompatible materials, including one or any of parylene-C, poly(2-hydroxyethyl methacrylate) (pHEMA), polydimethylsiloxane (PDMS), and polyvinyl acid (PGA). The array of flexible electrodes comprises 256 microelectrodes. Each of the plurality of insulation parylene-C layer comprises a plurality of perfusion holes. In addition, the plurality of flexible guidewires are attached to corner sections of the parylene-C layers of the array of flexible electrodes. The plurality of flexible guidewires are configured to be guided by external mechanical force such that the array of flexible electrodes traverse craniotomy and are unfurled by tracking the plurality of flexible guidewires. The plurality of flexible guidewires each comprises a steering tip and a pulling thread. The pulling thread is an absorbable surgical suture made of polyvinyl acid (PGA) and is capable of being absorbed by human body.

In certain embodiment of the subject invention, a method for deploying a guidewires-driven brain-computer interface (GD-BCI) system for minimally invasive implantation is provided. The method comprises guiding a plurality of flexible guidewires of the GD-BCI system and positioning an array of flexible electrodes of the GD-BCI system within a subdural or an epidural space, wherein the plurality of flexible guidewires are attached to the array of flexible electrodes. Each of the plurality of flexible guidewires is individually controlled by an external mechanical force through a first opening and exited through a second opening. Furthermore, when the pulling threads of the guidewires are directed out of the second opening, the array of flexible electrodes is positioned by drawing the pulling threads.

### BRIEF DESCRIPTION OF THE DRAWINGS

FIG. 1A shows a conceptual perspective view of an implanted device within the brain subdural space through four millimetre-scale skull windows to stimulate cortex and collect ECoG signals, FIG. 1B shows a fabricated GD-BCI system connected to a cranial pedestal which is compatible with Blackrock cereport headstage, wherein the GD-BCI system comprises three pulling threads with guidewires capable of navigating between dura mater and pia mater, and FIG. 1C shows an exploded illustrative view of the thin film electrodes array architecture, demonstrating an insulation parylene-C (DPX-C) layer, gold interconnect lines, and PEDOT:PSS/pHEMA electrodes, wherein the sensing area is 30 mm by 30 mm and has 256 channels, according to an embodiment of the subject invention.

FIG. 2A is a schematic representation of the steering tip guided inside subdural space, FIG. 2B is a schematic representation of the folded GD-BCI system implanted inside through a millimetre-scale skull window and deployed inside the brain by retracting the three pulling threads, and FIG. 2C shows photographic images of the GD-BCI system being deployed in a phantom brain model, according to an embodiment of the subject invention.

FIGS. 3A-3G show the implantation procedure of the GD-BCI on the right lateral cortex of the beagle dog. FIG. 3A is a optical image of the pre-treated beagle head with four skull openings. FIGS. 3B-3C show the steering procedure of the steering tip and the pulling thread.

FIGS. 3D-3E show the deployment procedure of the thin film electrodes array by tracking the three pulling thread. FIGS. 3F-3G show the implanted GD-BCI, according to an embodiment of the subject invention.

FIG. 4 shows the schematic of a real-time recording and decoding system with the GD-BCI device implanted in the brain of a subject under test. The signals are measured under different cortex regions, including frontal, motor, parietal, temporal lobes, according to an embodiment of the subject invention.

FIGS. SA-5B are photographic images of the phantom brain model and the implanted GD-BCI system, wherein FIG. 5A is a photographic image of the phantom brain model with an implanted GD-BCI system, having a scale bar of 25 mm, and FIG. 5B shows top-view images of the implanted GD-BCI system, having a scale bar of 20 mm, according to an embodiment of the subject invention.

FIG. 6 shows a load-deformation curve of the agarose gel, wherein the blue dot curve is the load-deformation curve of the agarose gel showing the mechanical characteristics of the micromechanical testing system, wherein the red curve is the fitting curve of the linear range of the blue curve, and wherein the fitted Young's Modulus is 4964 Pa, according to an embodiment of the subject invention.

FIGS. 7A-7H show the effect of perfusion openings in establishing an intimate contact with the brain phantom. FIGS. 7A-7B are photographic images of the thin film device with/without perfusion openings. FIG. 7C is an optical image of the agarose-gel-based artificial brain phantom. FIGS. 7D-7E show the GD-BCI without and with perfusion holes are placed on top of the brain phantom and gently tapped with a medical cotton ball in the same manner, wherein the red dashed circle is the position that the GD-BCI cannot form close contact with the gyri. FIG. 7F is an optical image of the red artificial cerebrospinal fluid on top of the brain phantom model. FIGS. 7G-7H show the GD-BCI without and with perfusion holes are placed on top of the brain phantom with CSF and gently tapped with a medical cotton ball in the same manner, wherein the yellow dashed circle is the position that CSF is trapped underneath the GD-BCI.

FIGS. 8A-8G show the epidural recording in canine models, wherein FIG. 8A shows the location of the deployed GD-BCI device on beagle cortex, the dark part being the temporal lobe and the light part being the occipital lobe; wherein FIGS. 8B-8C show signals recorded from a beagle dog during the sleep and awake status; wherein FIG. 8D shows the averaged power spectrum during the sleep and awake status; wherein FIG. 8E shows spectrograms of signals recorded by a representative channel in the sleep and awake status; wherein FIG. 8F shows spatial power spectral density (PSD) patterns in the alpha and gamma 1 band during the sleep and awake status, as well as the PSD difference between these two states; wherein FIG. 8G shows a channel-to-channel coherence map of different bands during the sleep and awake status, according to an embodiment of the subject invention.

### DETAILED DISCLOSURE OF THE INVENTION

Embodiments of the subject invention pertain to a guidewires-driven brain-computer interface (GD-BCI) system and methods for minimally invasive implantation.

The terminology used herein is for the purpose of describing particular embodiments only and is not intended to be limiting of the invention. As used herein, the term “and/or” includes any and all combinations of one or more of the associated listed items. As used herein, the singular forms “a,” “an,” and “the” are intended to include the plural forms as well as the singular forms, unless the context clearly indicates otherwise. It will be further understood that the terms “comprises” and/or “comprising,” when used in this specification, specify the presence of stated features, steps, operations, elements, and/or components, but do not preclude the presence or addition of one or more other features, steps, operations, elements, components, and/or groups thereof.

Unless otherwise defined, all terms (including technical and scientific terms) used herein have the same meaning as commonly understood by one having ordinary skill in the art to which this invention pertains. It will be further understood that terms, such as those defined in commonly used dictionaries, should be interpreted as having a meaning that is consistent with their meaning in the context of the relevant art and the present disclosure and will not be interpreted in an idealized or overly formal sense unless expressly so defined herein.

When the term “about” is used herein, in conjunction with a numerical value, it is understood that the value can be in a range of 90% of the value to 110% of the value, i.e. the value can be +/−10% of the stated value. For example, “about 1 kg” means from 0.90 kg to 1.1 kg.

In describing the invention, it will be understood that a number of techniques and steps are disclosed. Each of these has individual benefits and each can also be used in conjunction with one or more, or in some cases all, of the other disclosed techniques. Accordingly, for the sake of clarity, this description will refrain from repeating every possible combination of the individual steps in an unnecessary fashion. Nevertheless, the specification and claims should be read with the understanding that such combinations are entirely within the scope of the invention and the claims.

The utility of electrocorticographic brain-computer interface (ECoG-BCI) is firmly established in both clinical settings and basic neuroscience due to its high fidelity and non-penetrating nature. However, the existing BCI devices require compromises regarding coverage area, electrode density, surgical invasiveness, and potential complication risks, which do not fully meet the increasing demands of advanced BCI applications.

According to embodiments of the subject invention, a deployable bidirectional, guidewires-driven brain-computer interface (GD-BCI) system operated via mechanically controlled guidewires is provided. The GD-BCI system can be implanted into subdural space or an epidural space through several millimetre-scale craniotomies, offering a large coverage (for example, 9 cm²) and high electrode density (for example, 28.4 electrodes per cm²).

The GD-BCI system showcases its chronic recording and cortical electrical stimulation capabilities within the canine motor cortex. Demonstrated benefits include minimized cerebral damage and complication risks, enhanced ECoG signal quality, and improved bidirectional communication performance. These advancements hold the promise of expanding the range of patients and users who can benefit from the BCI technologies.

The GD-BCI system may be implanted into a subdural space or an epidural space via several millimetre-scale craniotomies. According to embodiments of the subject invention, the GD-BCI system comprises a high-density array of flexible PEDOT:PSS/pHEMA electrodes (for example, a density of 28.4 electrodes cm⁻²) covering a large surface area (for example, 9 cm²), and a plurality of, for example, three flexible guidewires. The guidewires, affixed to the corners of the flexible electrodes array, can be introduced into a subdural space or an epidural space and guided by the external mechanical force. The folded flexible electrode array can traverse the craniotomy and be unfurled by tracking the guidewires.

The thin, perforated electrode array ensures excellent adaptability and close contact with the contoured surfaces of the brain. Additionally, deploying the GD-BCI system within the motor cortex of a canine model demonstrates its ability for chronic neural signal recording, cortical electrical stimulation, and acute neural decoding. As a result, the GD-BCI system is engineered to advance the safe clinical application of BCI technologies, providing the necessary coverage and electrode density for high-performance BCIs while remaining compatible with established clinical neurosurgical practices.

Hence, the GD-BCI system is advantageous in utilizing guidewires for minimally invasive implantation of the electrode array, achieving high-density electrode configuration (28.4 electrodes per cm²), and providing an extensive coverage area (9 cm²) through small craniotomies. These features collectively represent a significant advancement over the existing technologies, presenting a unique combination of safety, precision, and effectiveness in brain-computer interfacing previously unattainable.

### Design of the GD-BCI System

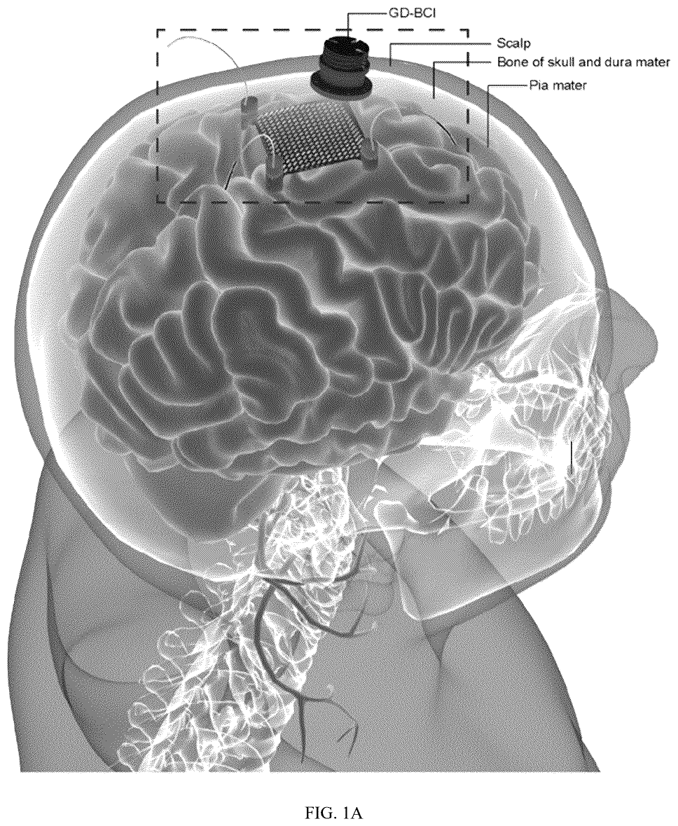

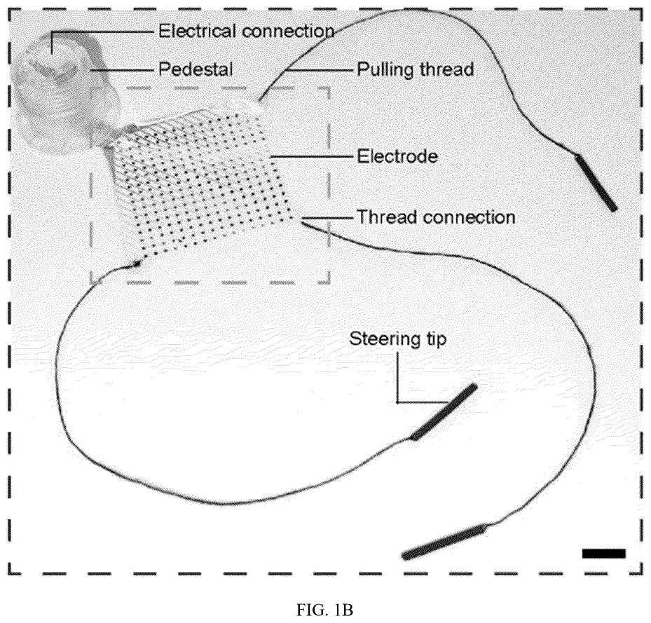

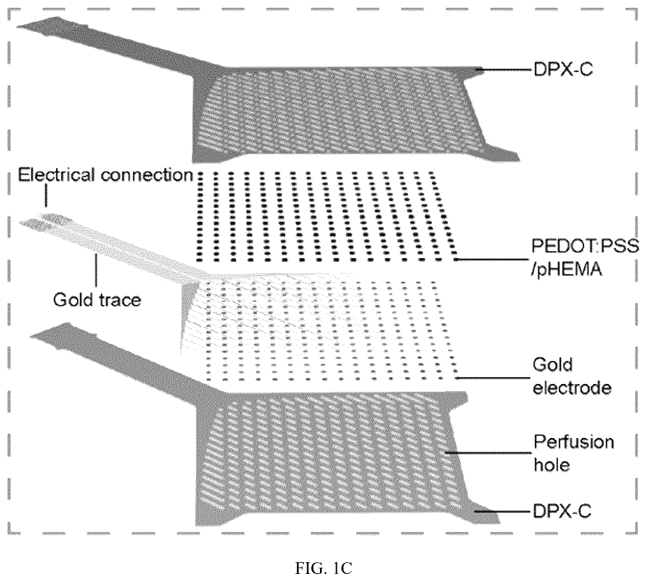

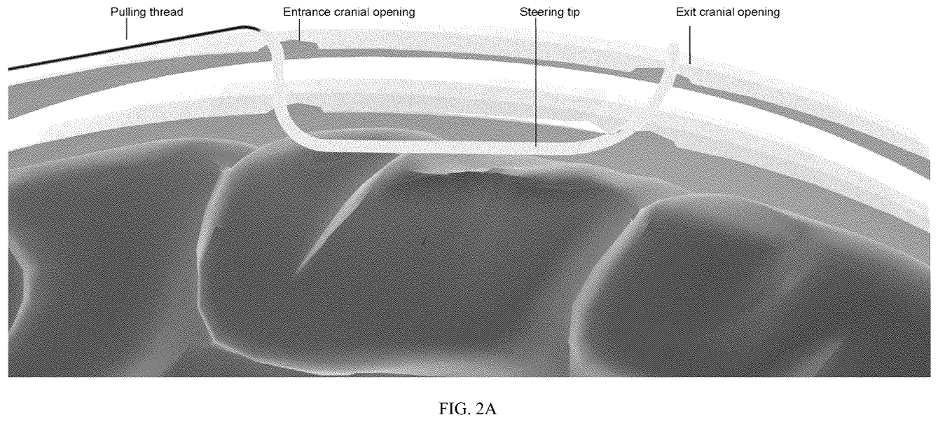

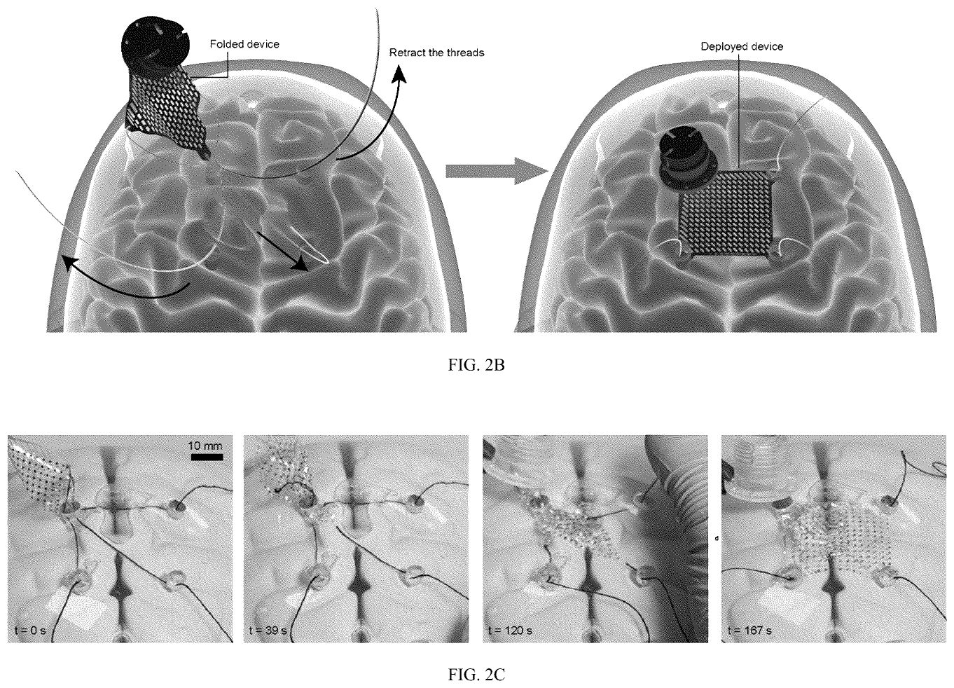

Referring to FIG. 1A, the GD-BCI system is engineered to achieve extensive cortical coverage and high-density recording/stimulation sites and designed for minimally invasive implantation over the pia mater through four millimetre-scale skull openings, significantly reducing implantation-related injuries. The patient-contact components of the GD-BCI system are constructed from entirely biocompatible materials, including parylene-C, poly(2-hydroxyethyl methacrylate) (pHEMA), polydimethylsiloxane (PDMS), and polyvinyl acid (PGA). Further, the GD-BCI system comprises two principal components: a thin film electrodes array as shown in FIG. 1C and guidewires as shown in FIG. 1C and FIG. 2A.

In one embodiment, the thin film electrodes array is designed to smoothly navigate the subdural space, conformally adhering to the curved surface of the brain. As shown in FIG. 1C, the array may incorporate, for example, 256 microelectrodes, embedded within a 10-μm-thick flexible parylene C sheet, covering a 9 cm² cortical area with a 2 mm pitch. The spatial resolution of the electrodes mitigates insufficient spatial sampling on human subdural ECOG signals and effectively decode human speech signals³³.

In one embodiment, PEDOT:PSS/pHEMA is deposited on gold microelectrodes having a diameter of about 0.7 mm using an electropolymerization method. The PEDOT:PSS/pHEMA electrodes can improve the charge injection capacitance (CIC) and reduce the interface impedance, while maintaining good biocompatibility and biostability, which is sufficiently fine to deliver current stimulation and record neural activity on the cortical surface.

In one embodiment, between the PEDOT:PSS/pHEMA electrodes and the electrical connection pads, gold traces that are 200 nm thick, 10 μm wide, 10 μm apart, 3 cm long, and fully encapsulated between two layers of parylene C (5 μm at the bottom and 5 μm at the top; as shown in FIG. 1C are routed. The electrical connection part is patterned in a land grid array (LGA) layout that can be precisely bonded on the connection pads of CerePort round PCB on the pedestal to minimise the whole size of the fix part on human head and reduce the surgical damage.

As shown in FIG. 1B, the parylene-C layer of the thin film electrodes array had extended sections at the three corners position for attaching the guidewires, which can be navigated inside the subdural space and deployed under the dura mater.

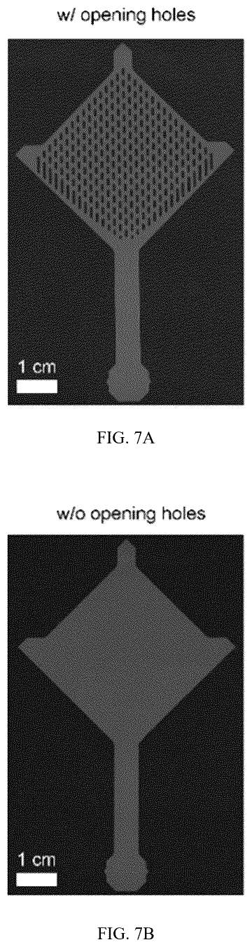

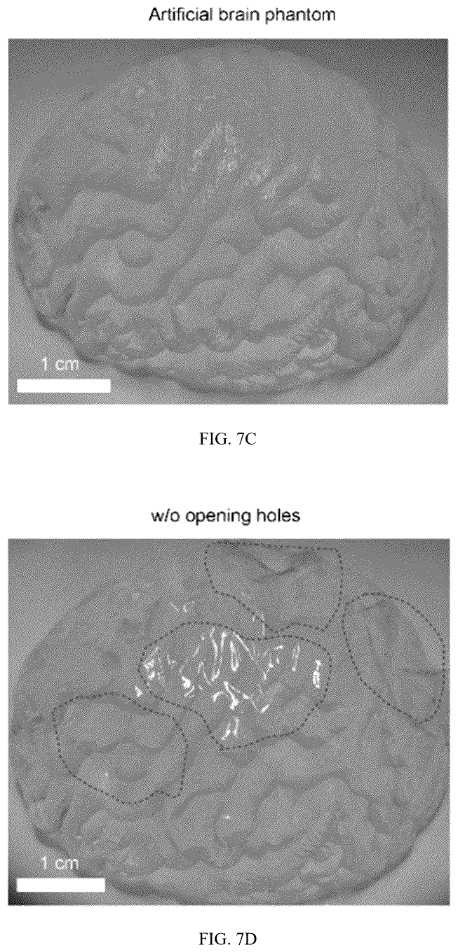

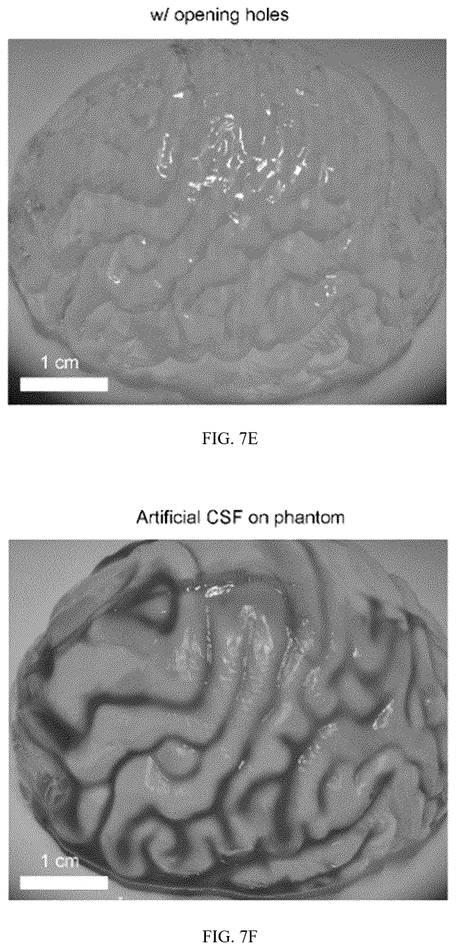

As shown in FIGS. 1A-1C and FIGS. 7A-7H, perfusion holes are patterned in the parylene C layer throughout the thin film. Although the device was as thin as 10 μm, some region of device cannot conform the structure of curved brain surface structures shown in FIG. 7D, and parts of cerebrospinal fluid (CSF) were commonly trapped between the tissue and the electrode shown in FIG. 7G. These challenges resulted in diminished consistency of signal recording, attributed to the emergence of ‘dead zones’ characterized by trapped CSF, potential air bubbles, and wrinkling of the device. The perfusion openings can enhance the stretchability of the device, enabling the thin film electrodes array to achieve a conformal fit and maintain a close interface with the convolutions of the brain's sulci and gyri shown in FIG. 7E. Furthermore, the openings can also help to perfuse cerebrospinal fluid (CSF) away from the electrode contacts. Therefore, an intimate interface between the GD-BCI and the surface of the brain was maintained to avoid volume conduction from the CSF shown in FIG. 7H.

### Deployment Demonstration in Brain Phantom

The deployment of the GD-BCI system includes two key steps: guiding the guidewires and positioning the thin film electrodes array within the subdural space or an epidural space. Each of the three guidewires is individually directed through a skull opening and exited through another, utilizing a surgical biocompatible guidewire. Referring to FIG. 2B and FIG. 2C, once all three pulling threads are navigated through one same skull hole and out through the three distinct openings, the thin film electrodes array is deployed by manually drawing the threads. The flexibility of the array allowed it to be folded prior to insertion into the subdural space or the epidural space. The mechanical force is applied on the corner of the thin film electrodes array via the threads. This action pulled most of the sensing area into the phantom gap, and subsequently, all three threads are drawn together to fully deploy the device across the cortex surface. The thin film electrodes array with perfusion holes could be attached conformally and maintain an intimate interface to the curved surface of the brain.

### In-Vivo Epidural Recording in Canine Models

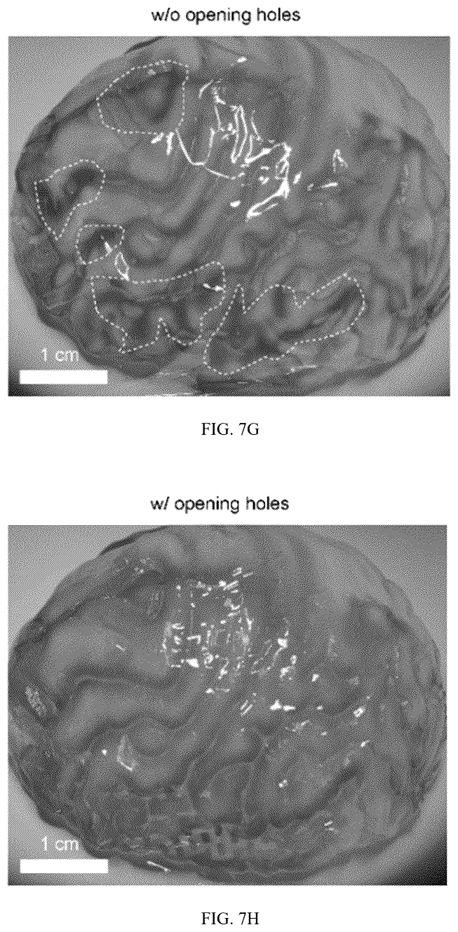

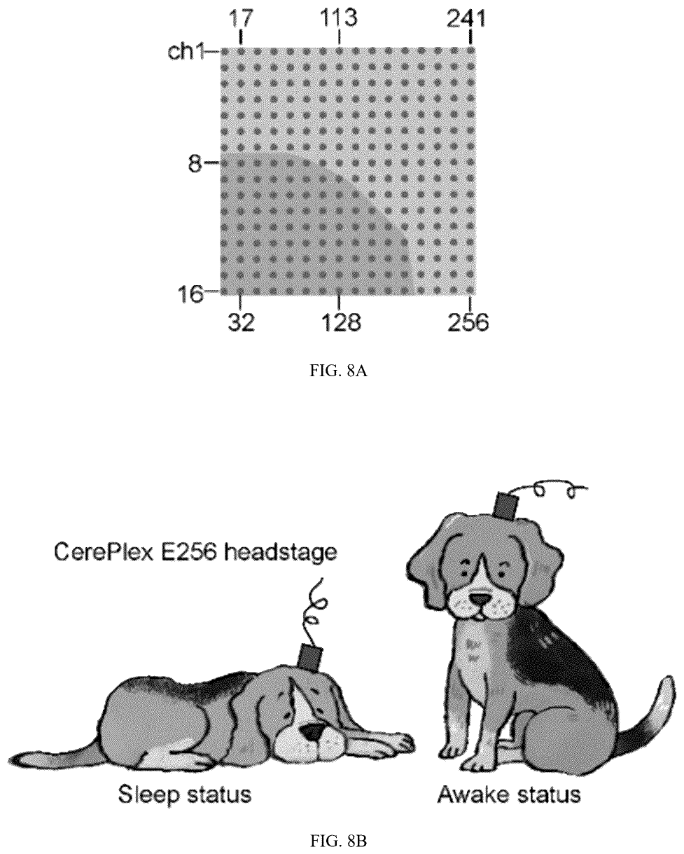

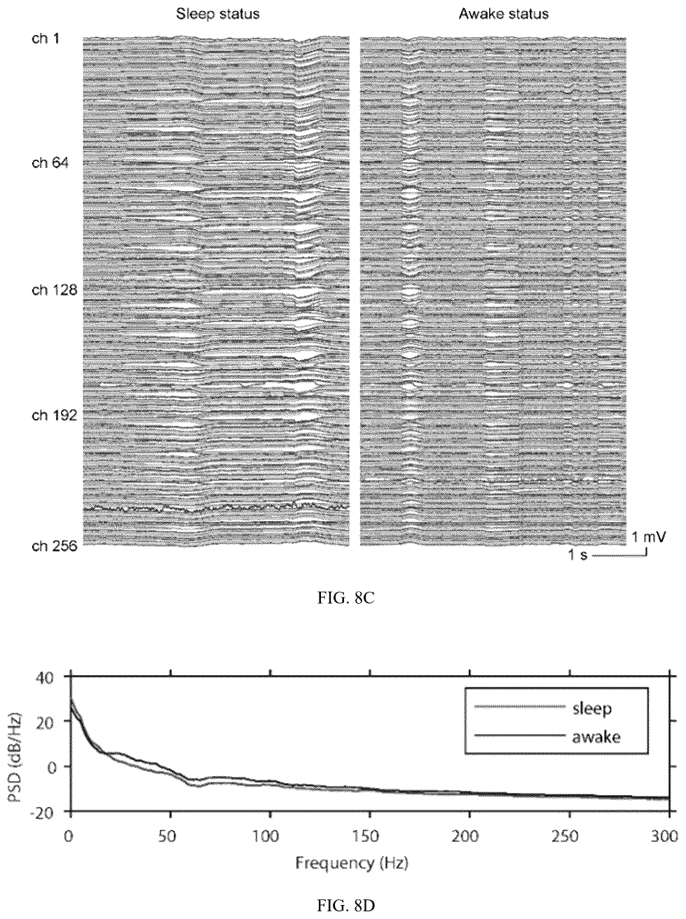

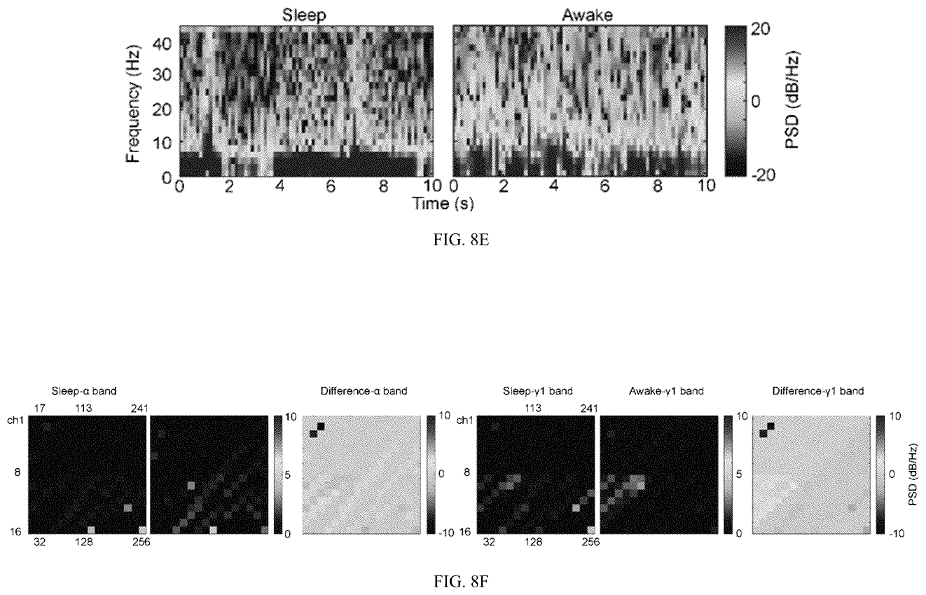

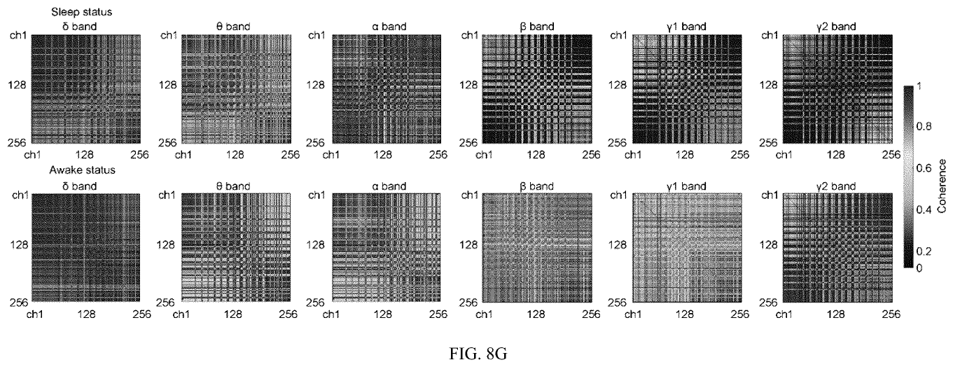

The GD-BCI recorded high-quality micro-ECoG signals during both sleep and awake states are shown in FIGS. 8B-8C. The mean spectrum across 256 channels indicates higher levels of alpha/beta (for example, 10-30 Hz) and gamma 1 band (for example, 30-50 Hz) power during the awake state compared to the sleep state as shown in FIG. 8D. Moreover, the power density spectrum for an individual channel confirms the increase in alpha/beta and gamma 1 oscillation power during wakefulness as shown in FIG. 8E. Additionally, the GD-BCI provides spatially resolved patterns of neural state changes that are illustrated by the power spectral density (PSD) maps in the alpha and gamma 1 bands across various channels as shown in FIG. 8F.

With the high spatiotemporal resolution and bandwidth, the epidural recordings from the GD-BCI facilitates the investigation of coupling between distributed brain regions under different states. The coupling between recording sites is assessed by coherence, a frequency-domain measure of linear association. The channel-to-channel coherence maps as shown in FIG. 8G reveal that the gamma band, including gamma 1 (for example, 30-50 Hz) and gamma 2 (for example, 50-100 Hz), exhibits the lowest coherence, while the delta band shows the highest coherence across both sleep and awake states. The disparity may arise from the fact that low-frequency signals can couple over longer distances, whereas high-frequency signals are more spatially constrained. Notably, the channel-to-channel coherence is higher in the awake state than in the sleep state, particularly from the theta band to gamma 2 band. One potential explanation for this phenomenon is the reduced level of consciousness during sleep, diminishing functional interactions in various cortical and subcortical regions and leading to alterations and disruptions in cortical integration during the information processing.

### Materials and Methods

### Deployment Demonstration in Brain Phantom

The deployment of the GD-BCI system involves two key steps: guiding the guidewires and positioning the thin film electrodes array within the subdural space or the epidural space. Each of the three guidewires is individually directed through a skull opening and exited through another within a custom-designed 3D printed brain phantom as shown in FIG. 2A.

Referring to FIGS. 2B and 2C, once all three pulling threads are navigated through one same skull hole and out through the three distinct openings, the thin film electrodes array is deployed by manually drawing the threads. The flexibility of the array allowed it to be folded prior to insertion into the subdural space or the epidural space. The mechanical force is applied on the corner of the thin film electrodes array via the threads. This action pulled most of the sensing area into the phantom gap, and subsequently, all three threads are drawn together to fully deploy the device across the cortex surface. The implanted GD-BCI system within the brain phantom is depicted in FIGS. 5A-5B. The thin film electrodes array with perfusion holes could be attached conformally and maintain an intimate interface to the curved surface of the brain.

### Fabrication Procedure of the Thin Film Electrodes Array

The thin film electrodes array is fabricated on a 4-inch Si wafer (Namkang Hi-Tech) using standards photolithography processing. The substrate is cleaned by O₂/SF₆ plasma under vacuum (10 mins at 200 W and 20 mT pressure with 30 sccm O₂ and 10 sccm SF₆) using a PlasmaPro 80 reactive ion etching (RIE) and followed by O₂ RIE (10 mins at 200 W and 20 mT pressure with 30 sccm O₂). The cleaned substrates are then immersed in 0.5% Octadecyltrichlorosilane (Sigma-Aldrich) with m-Xylene (Sigma-Aldrich) for 8 hours and washed with Chloroform (Sigma-Aldrich) afterwards to form a sacrificial OTS layer for the lateral mechanical peel off from the substrate. 5 μm thickness of parylene-C (DPX-C, SCS Company) is deposited by SCS Labcoater 2 for the bottom insulation layer. AZ NLOF 2020 photoresist (Microchem) is spin coated onto the DPX-C layer at 3000 rpm for 30 s. This is followed by a pre-baking step on a hot plate at 110° C. for 60 s (Apogee bake plate). After the substrates returned to ambient temperature, they are exposed to ultraviolet (i-line, 66 mJ cm⁻²) using a mask aligner (Karl Suss MA/BA6, vacuum contact), and this is followed by a post-baking step at 110° C. for 60 s. The substrates are allowed to cool for several minutes and developed in NMD238 (Microchem) for 30 s; this is then rinsed with DI water and air dried with compressed nitrogen. The Cr (20 nm thickness) and gold (200 nm thickness) are deposited on the patterned AZ NLOF 2020 photoresist using a Lesker thermal evaporator (under vacuum <6×10⁻⁶ torr) to produce the interconnect line. Liftoff is performed by dimethylsulfoxide (DMSO, Sigma-Aldrich). Following the second photolithography procedure to expose the shape and the perfusion holes of the device bottom layer shown in FIG. 1C using AZ NLOF 2070 (Microchem), spin coated for 1500 rpm for 60 s, and baked at 110° C. for 90 s. They are then UV-exposed (i-line) for 196 mJ cm⁻² and developed in NMD238 for 100 s. The substrate is then etched using RIE (Oxford Plasma Pro 100 RIE, 200 W/O2 30 sccm) until DPX-C exposure is complete. The device is then treated using a 0.5% silane solution (A174, SCS Company) and coated with 5 μm thickness of DPX-C. Following the third photolithography procedure to expose the microelectrodes, the electrical connection pads, and the perfusion holes of the device top layer shown in FIG. 1C using AZ NLOF 2070. The substrate is then etched using RIE until the metal and perfusion holes are completely exposed.

### Packaging of the GD-BCI System

The electrical connection part of the thin film electrodes array is patterned in a land grid array (LGA) layout that precisely matches the connection pads of CerePort round PCB on the pedestal (Blackrock). The electrical connection pads are bonded with the Cereport round PCB using an anisotropic conductive film (DP3342MS, Dexerials) by a hot-press procedure (150° C., 2 MPa, 6 s). The pedestal is compatible with Blackrock CerePlex headstage.

To deploy the GD-BCI system, three absorbable surgical sutures with the length of 10 cm are attached to the corners of the thin film electrodes worked as the pulling thread shown in FIG. 1B and FIGS. 2A-2C (R016-0, Jinhuan Medical Products Co., Ltd). The threads are attached using a medical-level UV cure adhesive (Loctite AA 3311) with 5 μL. Another side of the threads are attached with a 50 mm length flexible steering tips under the same condition.

Electropolymerization of PEDOT:PSS and pHEMA

The electropolymerization method using an electrochemical workstation (Gamry Reference 600+) has been previously reported³⁴. Aqueous solution of 3,4-Ethylenedioxythiophene (EDOT, Sigma-Aldrich, 0.01 M) and polystyrene sulfonate (PSS, Sigma-Aldrich, 2 wt %) is used to deposited PEDOT:PSS with current density of 4.7 mA cm⁻² and deposition time of 30 s on the metal microelectrodes based on a classical three-electrodes cell with the working electrode connected to the microelectrode on the device while the reference electrode connected to the Ag/AgCl electrode and counter electrode connected to a platinum pad (10×10 mm). Following by the pHEMA deposition. It is based on a similar three-electrode electrochemical cell setup, but with two distinct half-cells. The working electrode is connected to the electrode pad on the probe, with the Ag/AgCl reference electrode placed in the cathodic half-cell containing a solution of HEMA (0.1 M)/EGDMA (2 wt %)/(NH₄)₂S₂O₈ (0.1M). The counter electrode is linked to a platinum pad in the anodic half-cell filled with sulfuric acid (H₂SO₄, 0.025 M). The pHEMA deposition involved cyclic voltammetry ranging from 0 V to −0.9 V for 100 cycles at a scan rate of 100 mV s⁻¹. The samples are rinsed with DI water after each deposition step and then dried with nitrogen.

### Deployment Demonstration in Brain Phantom

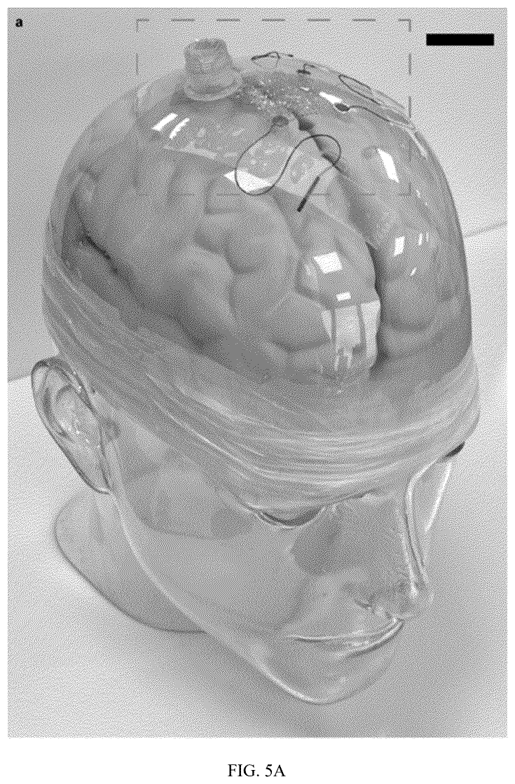

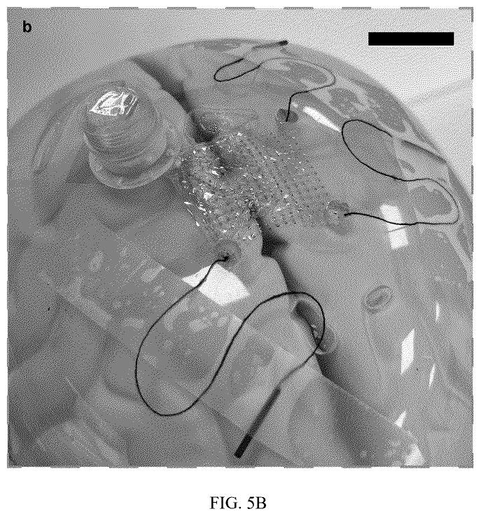

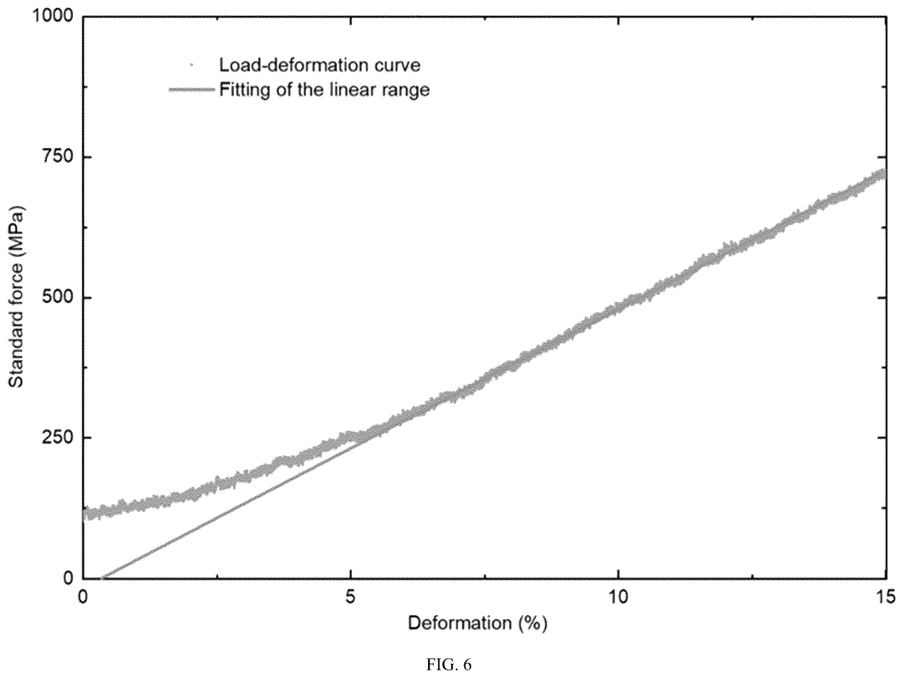

The brain phantom shown in FIG. 2C and FIGS. 5A-5B is fabricated by 3D printed resin based on a custom design. The minimal gap between the dura mater phantom (transparent) and the pia mater phantom (light pink) is 2 mm which is comparable with real human subdural space distance. To mimic the adipose tissue and connective tissue in human subdural space with Young's modulus of 1.6-5.5 KPa³⁵, a conductive agarose gel is injected into the phantom gap. The agarose gel is made by mixing 0.2 wt % agarose powder (MB755-0500, Bio-Helix Co., Ltd) in 10× Phosphate-buffered saline solution (PBS, Sigma-Aldrich) and heating up to 90 degrees for fully dissolving the powder. This is followed by injecting the solution into the phantom gap and then cooling in room temperature for the gel solidification. The Young's modulus of the artificial tissue gel is measured by exerting loads on a cube gel (2 cm on each side) with a tensile-compressive tester (Zwick Roell). As shown in FIG. 6, the load-displacement curve is translated to the stress strain curve for fitting the modulus, which is the slope of the curve.

The steering tips are steered manually. After all the three steering tips are navigated to the designed position, the GD-BCI system are deployed by manually pulling the threads shown in FIG. 2B and FIG. 2C.

### In-Vitro Electrical Performance Characterization

To indicate whether the microelectrodes and the interconnect lines are damaged during the implantation and deployment procedures, the three-point impedance measurements are taken before and after the implantation in brain phantom using an electrochemical workstation (Gamry Reference 600+) in 1×PBS.

The CSC and CIC characterization are taken for evaluating the electrical stimulation performance of the GD-BCI system. The CSC testing is carried out by three-point cyclic voltammetry measurement in 1×PBS solution using the electrochemical workstation with a 0.1V sweep rate and 2 mV step within the water electrolysis window of +0.6 V to −0.9 V. Six cycles are performed for each measurement to allow the recording to settle, and only the final recorded cycle is analyzed. The CSC can be calculated by the following relationship:

$$CSC = ∮ \frac{i}{vA} dV \qquad ( 2 )$$

where ν is the scan rate of the CV measurement, A is the area of the microelectrodes, i is the anodic current and V is the scanning potential.

The CIC testing is carried out by a charged balanced stimulation in 1×PBS solution in a two-electrodes configuration. A stimulator isolator (ISO-Flex, A.M.P.I., Israel) controlled by a Master-9 (A.M.P.I., Israel) is connected to the microelectrodes and provide a charge-balanced current stimulation pulse with a width of 200 μs, followed by a short pause of 50 μs, and then provide a second inverse pulse with a width of 400 μs and half amplitude of the first pulse. A digital Multimeter (Keithley DMM6500) is used to record the current and the voltage transient. The platinum pad is used as counter and reference electrodes. The current pulse amplitude is gradually increased until observing the cathodic or anodic voltage transient exceeds the water window. The CIC can be calculated by the following relationship:

$$CIC = \frac{{I}_{max} {t}_{pulse}}{A} \qquad ( 3 )$$

where Iₘₐₓ is the maximum current pulse amplitude, tₚᵤₗₛₑ is the first forward pulse width, and A is the microelectrode area.

To examine the signal quality recorded by the GD-BCI system, typical stimulated neural signals generated from a neural signal simulator (PN-8282, Blackrock) are conducted into the conductive agarose gel inside the brain phantom through a stainless wire, and the GD-BCI system is also deployed into the brain phantom to detect the simulated signals. The pedestal of the GD-BCI system is connected to a CerePlex headstage and a neural signal recording system (Cerebus R, Blackrock).

Electrical performance characterization is performed for n=4 thin film electrodes array with 1024 electrodes in total.

In-Vivo Signal Sensing with the ECOG Probe

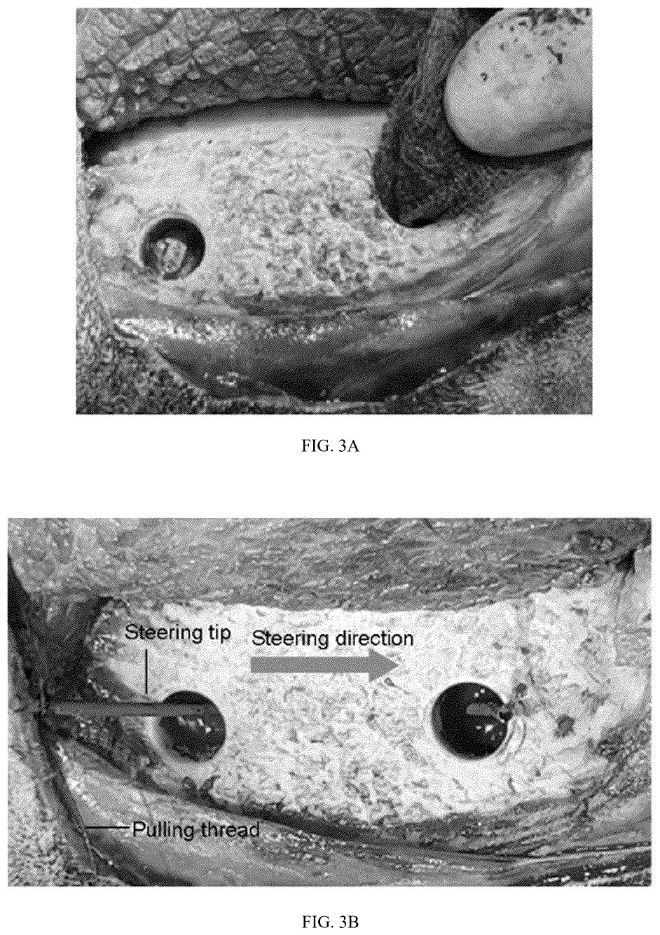

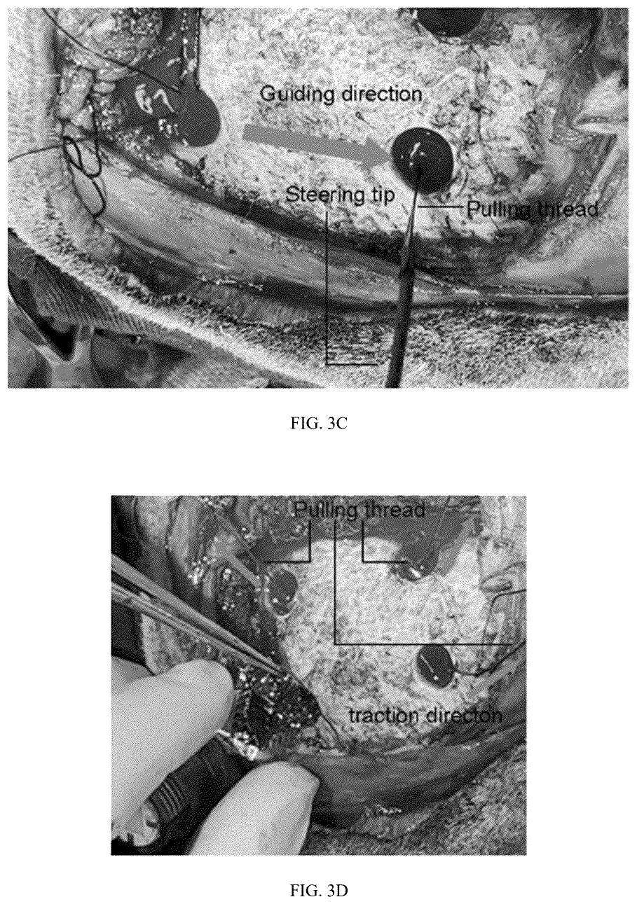

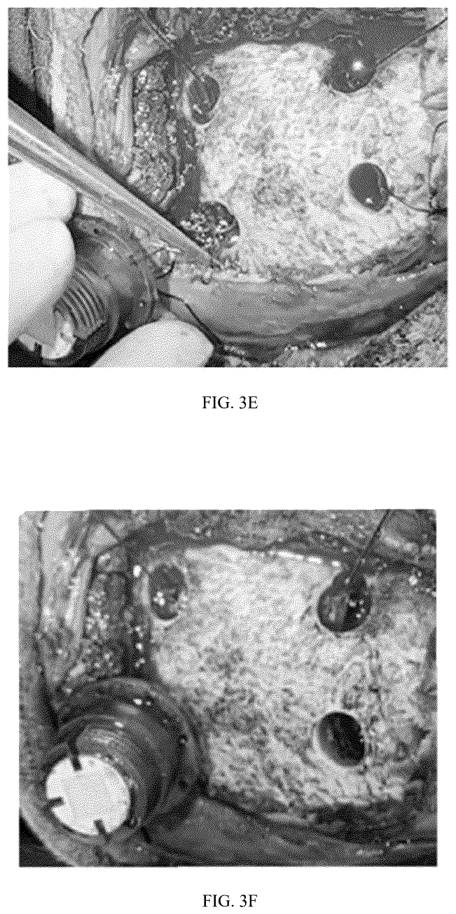

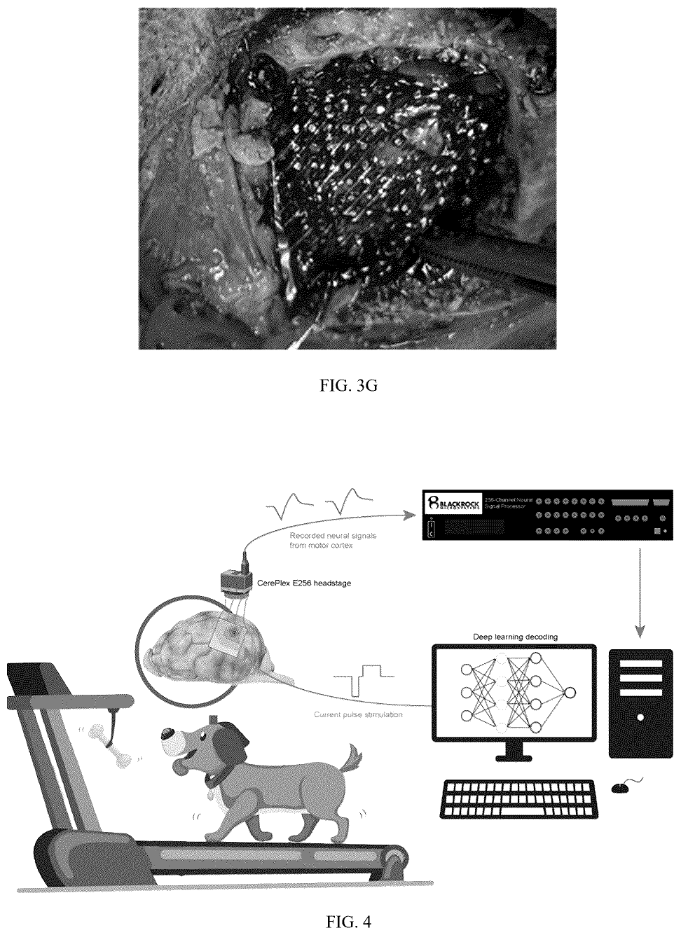

To evaluate the performance of the guidewires-driven minimal invasive ECoG system on reality animal operations, the implantation surgery is conducted on beagle dogs. Assisting by the CT images, the cranium of the right hemisphere is properly exposed. As shown in FIGS. 3A-3G, four cranial windows, each with a diameter of 5 mm, are drilled to construct a square shape with a side length of 35 mm, ensuring that the ECoG probe can be deployed flat on the cortex. Similar to the implantation method described in FIGS. 2A-2C, the ECoG probe is properly unfolded on the surface of the cortex including the frontal, motor, parietal, temporal lobes, guided by mechanical force applied to the steering tip as shown in FIGS. 3A-3F. To confirm the properties of the electrodes after deployment and all in-vivo testing, the skull was opened to show the fully deployed device as illustrated by FIG. 3G. The results above demonstrate that the guidewires-driven minimal invasive implantation method according to embodiments of the subject invention can be successfully conducted on large animals to deploy the thin film ECoG probe onto the target cortex region and reliably achieve signal recording.

To further evaluate the performance of the ECoG probe in sensing the signals on the subdural space or the epidural space, the dog was put on a running machine as well as connected with a CerePlex E256 headstage and a Cerebus neural signal processor. FIG. 4 shows the recording schematic of motion evoked event-related potential (ERP) signals recorded from cortex regions, including frontal, motor, parietal, temporal lobes. The recorded signals were processed and decoded by a deep learning model. The electrical stimulation can be performed on the GD-BCI to affect animal's movements according to the decoded signals.

### ECoG Signal Acquisition and Analysis

After the ECoG probe is deployed on the cortex, a CerePlex E256 headstages and the Neural Signal Processor (Cerebus System, Blackrock Microsystems, USA) were connected on the probe pedestal for signal recording. Lowpass 500 Hz and 50 Hz notch filters are applied to raw data to filter out the ECoG signal from background noise. The filtered signal is acquired and digitized at a sampling rate of 1 k Hz. The background noise level (root mean square) and peak-to-peak SNR are computed with self-written codes in Matlab.

Following are examples that illustrate procedures for practicing the invention. These examples should not be construed as limiting. All percentages are by weight and all solvent mixture proportions are by volume unless otherwise noted.

### Example 1—In-Vivo Minimal Invasive Implantation and Deployment of the GD-BCI System

The use of the dogs is approved by the ethics committee of the Guangzhou Huateng Biomedical Technology Co., LTD. (Guangdong, China). All procedures are performed under the approval of the Animal Research Ethics Sub-Committee of Guangzhou Huateng Biomedical technology Co., LTD and in accordance with the guidelines outlined in the Association for Assessment and Accreditation of Laboratory Animal Care International (AAALAC). Adult male Beagles dogs aging [24-26] months are used in this project. The dogs are kept in standard air humidity with a 12-h light/dark cycle and fed with Wellness Complete Health food twice daily from 10 am to 11 am and 5 μm to 6 μm and allowed free access to water ad libitum throughout the course of the experiments.

The dog is pre-induced anaesthesia with isoflurane and positioned on a stereotaxic apparatus. Once the surgery started, the dose of the isoflurane is reduced and maintained at a level that is sufficient to produce analgesia without suppressing electrocorticographic activity (1.5-2%, flow speed, modal of the anaesthesia machine). Heart rate, blood pressure, body temperature, peripheral capillary oxygen saturation (SpO₂), and end-tidal carbon dioxide (ETCO₂) are monitored throughout the operation using an electrocardiogram monitor (BLT Q5 VET, Guangdong Biolight Meditech Co., Ltd.). The skin and muscle over the right hemisphere are crosscut to expose the skull. The precise location of the target region is determined based on the dog brain atlas³⁶ and the brain CT scanning images captured before craniotomy. According to the dimension of the ECoG probe (3 cm×3 cm), four cranial windows with 5 mm diameter are drilled at four corners of a 3.5 cm by 3.5 cm square covering most of the right hemisphere to ensure the deployment of the probe. The three guidewires combined on the three corners of the ECoG probe are inserted into one same hole. With the leading of the mechanical force applied manually, the guidewires are steered out of the other three holes one by one. After all the three guidewires are navigated out of the three holes, a pulling force (1 N) is further applied on the threads to deploy the ECoG probe on the subdural space manually. The force applied on the threads is controlled by dynamometer (Chengying Sensors Co., Ltd.). Finally, the pulling threads are cut after the probe is unfolded properly on the surface of the cortex. The reference and ground of the probe is connected to a skull screw using silver wires.

The GD-BCI system is a minimally invasive system that can be implanted through tiny openings in the skull. The system employs guidewires to accurately place a dense array of electrodes over a large area of the brain. These electrodes, exceptionally numerous and flexible, adapt to the brain's surface to record and stimulate brain activity with high precision. Designed for treating neurological conditions and advancing neuroscience research, the GD-BCI system minimizes risks associated with the traditional brain surgeries such as tissue damage and infection, making it safer and more acceptable for patients.

Accordingly, the GD-BCI system solves the long-standing issue of invasiveness and safety in brain-computer interfaces. The traditional methods require large cranial openings, increasing the risk of complications such as tissue damage, swelling, and infection. The GD-BCI system offers an alternative solution by facilitating extensive brain coverage and high electrode density through minimally invasive procedures, addressing the critical need for safer, yet effective, brain mapping and stimulation tools in both clinical and research settings.

By utilizing guidewires to implant a densely packed electrode array into the brain via small, millimeter-scale craniotomies, the GD-BCI system significantly reduces surgical risks compared to the traditional brain surgeries. The high-density electrode array (for example, 28.4 electrodes per cm²) spread over a large area (for example, 9 cm²) enables precise neural signal recording and stimulation, ensuring both safety and efficacy in interfacing with the brain.

By combining an advanced electrode array design with a unique, minimally invasive deployment technique, the GD-BCI system represents a significant improvement to the existing brain-computer interfaces implantation method. The advantages over the existing solutions include reduced surgical risks, enhanced brain coverage, and improved electrode density for more accurate brain signal mapping, making it a superior choice for neurological research and treatment, offering a safer and more effective alternative for patients and researchers.

Embodiment 1. A guidewires-driven brain-computer interface (GD-BCI) system for minimally invasive implantation, comprising:
an array of flexible electrodes; and
a plurality of flexible guidewires attached to the array of flexible electrodes.

Embodiment 2. The GD-BCI system of Embodiment 1, wherein the array of flexible electrodes comprises a plurality of insulation parylene-C layers, metal interconnect lines, and electrodes made of PEDOT:PSS/pHEMA and gold.

Embodiment 3. The GD-BCI system of Embodiment 1, wherein the adjacent metal interconnect lines are spaced apart by a distance of 10 μm.

Embodiment 4. The GD-BCI system of Embodiment 2, wherein the metal interconnect lines are formed of gold and fully encapsulated between adjacent insulation parylene-C layers.

Embodiment 5. The GD-BCI system of Embodiment 1, wherein the array of flexible electrodes has a density of 28.4 electrodes cm⁻².

Embodiment 6. The GD-BCI system of Embodiment 1, wherein the plurality of flexible guidewires have three steering tips.

Embodiment 7. The GD-BCI system of Embodiment 1, wherein the plurality of flexible guidewires are configured to be guided by an external mechanical force such that the array of flexible electrodes traverse craniotomy and are unfurled by tracking the plurality of steering tips.

Embodiment 8. The GD-BCI system of Embodiment 1, wherein the array of flexible electrodes is formed of biocompatible materials, including one or any of parylene-C, poly(2-hydroxyethyl methacrylate) (pHEMA), polydimethylsiloxane (PDMS), and polyvinyl acid (PGA).

Embodiment 9. The GD-BCI system of Embodiment 1, wherein the array of flexible electrodes comprises 256 microelectrodes.

Embodiment 10. The GD-BCI system of Embodiment 2, wherein each of the plurality of insulation parylene-C layers comprises a plurality of perfusion holes.

Embodiment 11. The GD-BCI system of Embodiment 2, wherein the plurality of flexible guidewires are attached to corner sections of the parylene-C layers of the array of flexible electrodes.

Embodiment 12. The GD-BCI system of Embodiment 1, wherein the plurality of flexible guidewires each comprises a steering tip and a pulling thread.

Embodiment 13. The GD-BCI system of Embodiment 12, wherein the pulling thread is an absorbable surgical suture made of polyvinyl acid (PGA) and is capable of being absorbed by a human body.

Embodiment 14. A method for deploying a guidewires-driven brain-computer interface (GD-BCI) system for minimally invasive implantation in a subject, the method comprising:
obtaining a GD-BCI system according to embodiment 1;
guiding the plurality of flexible guidewires of the GD-BCI system; and
positioning the array of flexible electrodes of the GD-BCI system within a subdural space or an epidural space of a subject.

Embodiment 15. The method of Embodiment 14, wherein the guidewires are individually controlled manually through a first opening in the subject and exited through a second opening in the subject.

Embodiment 16. The method of Embodiment 15, wherein when the pulling threads of the guidewires are directed out of the three openings respectively, the array of flexible electrodes is positioned by drawing the pulling threads.

Embodiment 17. The method of embodiment 14, wherein when the array of flexible electrodes of the GD-BCI system is positioned within an epidural space of a subject, configuring the GD-BCI system to generate spectra to indicate different levels of alpha/beta (for example, 10-30 Hz) and gamma 1 band (for example, 30-50 Hz) power during an awake state and a sleep state of the subject.

Embodiment 18. The method of embodiment 17, wherein the alpha/beta band is in a range between 10 and 30 Hz.

Embodiment 19. The method of embodiment 17, wherein the gamma 1 band is in a range between 30 and 50 Hz.

All patents, patent applications, provisional applications, and publications referred to or cited herein are incorporated by reference in their entirety, including all figures and tables, to the extent they are not inconsistent with the explicit teachings of this specification.

It should be understood that the examples and embodiments described herein are for illustrative purposes only and that various modifications or changes in light thereof will be suggested to persons skilled in the art and are to be included within the spirit and purview of this application. In addition, any elements or limitations of any invention or embodiment thereof disclosed herein can be combined with any and/or all other elements or limitations (individually or in any combination) or any other invention or embodiment thereof disclosed herein, and all such combinations are contemplated with the scope of the invention without limitation thereto.

### REFERENCES

ADDIN EN.REFLIST 1 Chen, X., Wang, F., Fernandez, E. & Roelfsema, P. R. Shape perception via a high-channel-count neuroprosthesis in monkey visual cortex. Science 370, 1191-1196, doi:doi:10.1126/science.abd7435 (2020).
2 Bosking, W. H., Beauchamp, M. S. & Yoshor, D. Electrical Stimulation of Visual Cortex: Relevance for the Development of Visual Cortical Prosthetics. Annual Review of Vision Science 3, 141-166, doi:10.1146/annurev-vision-111815-114525 (2017).
3 Niketeghad, S. et al. Phosphene perceptions and safety of chronic visual cortex stimulation in a blind subject. Journal of neurosurgery 132, 2000-2007 (2019).
4 Winawer, J. & Parvizi, J. Linking electrical stimulation of human primary visual cortex, size of affected cortical area, neuronal responses, and subjective experience. Neuron 92, 1213-1219 (2016).
5 Flesher, S. N. et al. A brain-computer interface that evokes tactile sensations improves robotic arm control. Science 372, 831-836, doi:doi:10.1126/science.abd0380 (2021).
6 Schiefer, M., Tan, D., Sidek, S. M. & Tyler, D. J. Sensory feedback by peripheral nerve stimulation improves task performance in individuals with upper limb loss using a myoelectric prosthesis. Journal of neural engineering 13, 016001 (2015).
7 Colletti, V. et al. The First Successful Case of Hearing Produced by Electrical Stimulation of the Human Midbrain. Otology & Neurotology 28, 39-43, doi:10.1097/01.mao.0000247808.47712.02 (2007).
8 Atencio, C. A., Shih, J. Y., Schreiner, C. E. & Cheung, S. W. Primary auditory cortical responses to electrical stimulation of the thalamus. Journal of Neurophysiology 111, 1077-1087, doi:10.1152/jn.00749.2012 (2014).
9 Morrell, M. J. Responsive cortical stimulation for the treatment of medically intractable partial epilepsy. Neurology 77, 1295-1304 (2011).
10 Lee, B. et al. A single-center experience with the NeuroPace RNS system: a review of techniques and potential problems. World neurosurgery 84, 719-726 (2015).
11 Krauss, J. K. et al. Technology of deep brain stimulation: current status and future directions. Nature Reviews Neurology 17, 75-87 (2021).
12 Schalk, G. & Leuthardt, E. C. Brain-Computer Interfaces Using Electrocorticographic Signals. IEEE Reviews in Biomedical Engineering 4, 140-154, doi:10.1109/RBME.2011.2172408 (2011).
13 Caldwell, D. J., Ojemann, J. G. & Rao, R. P. Direct electrical stimulation in electrocorticographic brain-computer interfaces: Enabling technologies for input to cortex. Frontiers in Neuroscience 13, 804 (2019).
14 Khodagholy, D. et al. NeuroGrid: recording action potentials from the surface of the brain. Nature Neuroscience 18, 310-315, doi:10.1038/nn.3905 (2015).
15 Willett, F. R., Avansino, D. T., Hochberg, L. R., Henderson, J. M. & Shenoy, K. V. High-performance brain-to-text communication via handwriting. Nature 593, 249-254, doi:10.1038/s41586-021-03506-2 (2021).
16 Metzger, S. L. et al. A high-performance neuroprosthesis for speech decoding and avatar control. Nature, 1-10 (2023).
17 Ghanbari, L. et al. Cortex-wide neural interfacing via transparent polymer skulls. Nature Communications 10, 1500, doi:10.1038/s41467-019-09488-0 (2019).
18 Duraivel, S. et al. High-resolution neural recordings improve the accuracy of speech decoding. Nature Communications 14, 6938, doi:10.1038/s41467-023-42555-1 (2023).
19 Chang, E. F. Towards large-scale, human-based, mesoscopic neurotechnologies. Neuron 86, 68-78 (2015).
20 Shoaran, M., Pollo, C., Leblebici, Y. & Schmid, A. in 2012 Annual International Conference of the IEEE Engineering in Medicine and Biology Society. 5150-5153.
21 Tchoe, Y. et al. Human brain mapping with multithousand-channel PtNRGrids resolves spatiotemporal dynamics. Science Translational Medicine 14, eabj1441, doi:doi:10.1126/scitranslmed.abj1441 (2022).
22 Kaiju, T., Inoue, M., Hirata, M. & Suzuki, T. High-density mapping of primate digit representations with a 1152-channel μECoG array. Journal of Neural Engineering 18, 036025 (2021).
23 Hatashita, S., Koike, J., Sonokawa, T. & Ishii, S. Cerebral edema associated with craniectomy and arterial hypertension. Stroke 16, 661-668, doi:doi:10.1161/01.STR.16.4.661 (1985).
24 Mack, M. J. Minimally invasive and robotic surgery. Jama 285, 568-572 (2001).
25 Craniotomy: True Sham for Traumatic Brain Injury, or a Sham of a Sham? Journal of Neurotrauma 28, 359-369, doi:10.1089/neu.2010.1427 (2011).
26 Lin, C., Zhao, X. & Sun, H. Analysis on the risk factors of intracranial infection secondary to traumatic brain injury. Chinese Journal of Traumatology 18, 81-83 (2015).
27 Smalley, E. The business of brain-computer interfaces. Nature Biotechnology 37, 978-982, doi:10.1038/s41587-019-0231-y (2019).
28 Lebedev, M. A. & Nicolelis, M. A. Brain-machine interfaces: From basic science to neuroprostheses and neurorehabilitation. Physiological reviews 97, 767-837 (2017).
29 Song, S., Fallegger, F., Trouillet, A., Kim, K. & Lacour, S. P. Deployment of an electrocorticography system with a soft robotic actuator. Science Robotics 8, eadd1002 (2023).
30 Jeong, U.-J. et al. A minimally invasive flexible electrode array for simultaneous recording of ECoG signals from multiple brain regions. Lab on a Chip 21, 2383-2397 (2021).
31 Ho, E. et al. The layer 7 cortical interface: a scalable and minimally invasive brain-computer interface platform. bioRxiv, 2022.2001. 2002.474656 (2022).
32 Yamakawa, T. et al. Subdural Electrocorticogram Measurement with a Minimally-Invasive Procedure Using an SMA-Manipulated Microelectrode Array. Advanced Materials Research 222, 313-317, doi:10.4028/www.scientific.net/AMR.222.313 (2011).
33 Branco, M. P., Geukes, S. H., Aarnoutse, E. J., Ramsey, N. F. & Vansteensel, M. J. Nine decades of electrocorticography: A comparison between epidural and subdural recordings. European Journal of Neuroscience 57, 1260-1288, doi:https://doi.org/10.1111/ejn.15941 (2023).
34 Chik, G. K. K. et al. Flexible Multichannel Neural Probe Developed by Electropolymerization for Localized Stimulation and Sensing. Advanced Materials Technologies 7, 2200143, doi:https://doi.org/10.1002/admt.202200143 (2022).
35 Guimarães, C. F., Gasperini, L., Marques, A. P. & Reis, R. L. The stiffness of living tissues and its implications for tissue engineering. Nature Reviews Materials 5, 351-370, doi:10.1038/s41578-019-0169-1 (2020).
36 Johnson, P. J. et al. Stereotactic Cortical Atlas of the Domestic Canine Brain. Scientific Reports 10, 4781. doi:10.1038/s41598-020-61665-0 (2020).

We claim:

1. A guidewires-driven brain-computer interface (GD-BCI) system for minimally invasive implantation, comprising:
an array of flexible electrodes; and
a plurality of flexible guidewires attached to the array of flexible electrodes.

2. The GD-BCI system of claim 1, wherein the array of flexible electrodes comprises a plurality of insulation parylene-C layers, metal interconnect lines, and electrodes made of PEDOT:PSS/pHEMA and gold.

3. The GD-BCI system of claim 1, wherein the adjacent metal interconnect lines are spaced apart by a distance of 10 μm.

4. The GD-BCI system of claim 2, wherein the metal interconnect lines are formed of gold and fully encapsulated between adjacent insulation parylene-C layers.

5. The GD-BCI system of claim 1, wherein the array of flexible electrodes has a density of 28.4 electrodes cm⁻².

6. The GD-BCI system of claim 1, wherein the plurality of flexible guidewires have three steering tips.

7. The GD-BCI system of claim 1, wherein the plurality of flexible guidewires are configured to be guided by an external mechanical force such that the array of flexible electrodes traverse craniotomy and are unfurled by tracking the plurality of steering tips.

8. The GD-BCI system of claim 1, wherein the array of flexible electrodes is formed of biocompatible materials, including one or any of parylene-C, poly(2-hydroxyethyl methacrylate) (pHEMA), polydimethylsiloxane (PDMS), and polyvinyl acid (PGA).

9. The GD-BCI system of claim 1, wherein the array of flexible electrodes comprises 256 microelectrodes.

10. The GD-BCI system of claim 2, wherein each of the plurality of insulation parylene-C layers comprises a plurality of perfusion holes.

11. The GD-BCI system of claim 2, wherein the plurality of flexible guidewires are attached to corner sections of the parylene-C layers of the array of flexible electrodes.

12. The GD-BCI system of claim 1, wherein the plurality of flexible guidewires each comprises a steering tip and a pulling thread.

13. The GD-BCI system of claim 12, wherein the pulling thread is an absorbable surgical suture made of polyvinyl acid (PGA) and is capable of being absorbed by a human body.

14. A method for deploying a guidewires-driven brain-computer interface (GD-BCI) system for minimally invasive implantation in a subject, the method comprising:
obtaining a GD-BCI system according to claim 1;
guiding the plurality of flexible guidewires of the GD-BCI system; and
positioning the array of flexible electrodes of the GD-BCI system within a subdural space or an epidural space of a subject.

15. The method of claim 14, wherein the guidewires are individually controlled manually through a first opening in the subject and exited through a second opening in the subject.

16. The method of claim 15, wherein the plurality of flexible guidewires each comprises a steering tip and a pulling thread.

17. The method of claim 16, wherein when the pulling threads of the guidewires are directed out of the second opening, the array of flexible electrodes is positioned by drawing the pulling threads.

18. The method of claim 14, wherein when the array of flexible electrodes of the GD-BCI system is positioned within an epidural space of a subject, configuring the GD-BCI system to generate spectra to indicate different levels of alpha/beta (for example, 10-30 Hz) and gamma 1 band (for example, 30-50 Hz) power during an awake state and a sleep state of the subject.

19. The method of claim 18, wherein the alpha/beta band is in a range between 10 and 30 Hz.

20. The method of claim 18, wherein the gamma 1 band is in a range between 30 and 50 Hz.
